(19) United States
(12) Patent Application Publication
(10) Pub. No.: US 2013/0142732 A1
(43) Pub. Date: Jun. 6, 2013

(54) RECOMBINANT FLUORESCENT NANOPARTICLES

(71) Applicants: Jee-Won Lee, Seoul (KR); Keum-Young Ahn, Ansan-Si (KR); Jin-Seung Park, Incheon (KR)

(72) Inventors: Jee-Won Lee, Seoul (KR); Keum-Young Ahn, Ansan-Si (KR); Jin-Seung Park, Incheon (KR)

(73) Assignee: KOREA UNIVERSITY RESEARCH AND BUSINESS FOUNDATION, SEOUL (KR)

(21) Appl. No.: 13/487,921

(22) Filed: Jun. 4, 2012

(30) Foreign Application Priority Data
Dec. 2, 2011 (KR) 10-2011-0128615

(51) Int. Cl. C07K 19/00; A61K 49/00; G01N 21/64; B82Y 15/00

(52) U.S. Cl. 424/9.6; 530/350; 530/402; 530/358; 436/501; 435/7.92; 977/795; 977/920; 977/927

(57) ABSTRACT

A recombinant fluorescent protein nanoparticle having high fluorescence intensity and a method of detecting a target material using the same are provided. The protein nanoparticle has higher fluorescence intensity than a fluorescent protein, and is resistant to denaturation of the fluorescent protein at room temperature, thereby having higher structural stability than the fluorescent protein itself. In addition, since a self-assembled protein is used as a fusion partner of the fluorescent protein, the protein nanoparticle is biocompatible and safe. Moreover, when a linker peptide is additionally inserted into the protein nanoparticle, a suitable distance between the self-assembled protein and the fluorescent protein is maintained, thereby considerably increasing fluorescence intensity of the protein nanoparticle. The probe-binding protein nanoparticle can control distances between the fluorescent proteins on the surface thereof, thereby maximizing fluorescence intensity.

### CROSS-REFERENCE TO RELATED APPLICATION

This application claims priority to and the benefit of Korean Patent Application No. 2011-0128615, filed Dec. 2, 2011, the disclosure of which is incorporated herein by reference in its entirety.

### BACKGROUND

1. Field of the Invention

The present invention relates to a recombinant fluorescent protein nanoparticle having high fluorescence intensity, and a method of detecting a target material using the same.

2. Discussion of Related Art

A fluorescent protein is applied to and studied in various fields as an optical receptor amplifying a signal. However, the fluorescent protein generally has low detection sensitivity due to relatively low fluorescence intensity. To solve this problem, a variety of research aimed at increasing the fluorescence intensity of the fluorescent protein by gene mutation or formation of a fluorescent protein complex is being conducted, but the fluorescent protein still does not have a high level of fluorescence intensity.

### SUMMARY OF THE INVENTION

The present invention is directed to providing a protein nanoparticle to which a fluorescent protein having superior fluorescence intensity, structural stability and biocompatibility is fused.

One aspect of the present invention provides a protein nanoparticle in which a fluorescent protein is fused to a self-assembled protein and located at an outside of the fusion protein.

In one embodiment of the present invention, the protein nanoparticle may further include a linker peptide between the self-assembled protein and the fluorescent protein.

In one embodiment of the present invention, the self-assembled protein may be a human-derived self-assembled protein.

In one embodiment of the present invention, the self-assembled protein may be ferritin. Preferably, the self-assembled protein is a ferritin medium-chain protein.

Meanwhile, the linker peptide may be any one that can link the self- assembled protein to the fluorescent protein. In one embodiment, the linker peptide may include glycine.

In addition, a kind of the fluorescent protein according to the present invention is not specifically limited.

The present invention provides another protein nanoparticle in which a probe is bound to the protein nanoparticle. In one embodiment, the probe may be an aptamer.

The present invention is also directed to providing a biosensor including the probe-binding protein nanoparticle.

The present invention is also directed to providing a method of detecting a target material including confirming whether the probe of the protein nanoparticle reacts with a target material. In one embodiment, the method may be performed in vitro or in vivo.

The protein nanoparticle according to the present invention has much superior fluorescence intensity and superior structural stability since it is resistant to denaturation of the fluorescent protein at a room temperature, compared with the fluorescent protein itself. In addition, since the self-assembled protein is used as a fusion partner of the fluorescent protein, the protein nanoparticle is biocompatible and safe. Moreover, when the linker peptide is further inserted into the protein nanoparticle according to the present invention, a suitable distance between the self-assembled protein and the fluorescent protein is maintained, and thus the fluorescence intensity of the protein nanoparticle is considerably increased.

In addition, the probe-binding protein nanoparticle according to the present invention maximizes fluorescence intensity by controlling distances between the fluorescent proteins on a surface thereof.

### BRIEF DESCRIPTION OF THE DRAWINGS

FIG. 1 is schematics of the various gene fusions for synthesizing fluorescent proteins (eGFP and DsRed) and a ferritin nanoparticle, wherein (a) to (c) are genes in which a green fluorescent protein, eGFP, is fused with a ferritin nanoparticle, (d) and (e) are genes in which a red fluorescent protein, DsRed, is fused with a ferritin nanoparticle, (b), (c) and (e) are fusion genes including a glycine-rich linker peptide between a human ferritin heavy chain (hFTN-H) and each of the fluorescent proteins (eGFP and DsRed), and (a) and (d) are fusion genes not including a glycine-rich linker peptide.

FIG. 2A is schematics of the eGFP-fused ferritin nanoparticles according to the present invention, FIG. 2B shows TEM images and histograms for the nanoparticles, and FIG. 2C is a graph showing results of fluorescence emission analysis for the particles.

FIG. 3A shows the DsRed-fused ferritin nanoparticles according to the present invention, FIG. 3B shows TEM images and histograms for the nanoparticles, and FIG. 3C is a graph showing results of fluorescence emission analysis for the particles.

FIG. 4 is a graph showing results of fluorescence emission analysis for the eGFP-fused ferritin nanoparticles according to time.

FIG. 5 shows results of PDGF-BB analysis using DNA-aptamer-gFFNP, FIG. 5A is a schematic diagram illustrating an aptamer-based biomolecular detecting method, FIG. 5B is a graph showing results of detecting PDGF-BB present in PBS buffer using DNA-aptamer-conjugated gFFNPs, DNA-aptamer-conjugated eGFP and DNA-aptamer-conjugated Cy3 as reporter probes, and FIG. 5C is a graph showing a linear correlations based on a linearized form of the Langmuir absorption isotherm.

FIG. 6 is a graph showing results of PDGF-BB analysis in a biological sample using biotin-linked DNA-aptamer-conjugated gFFNP,

FIG. 6A is a graph showing results of assay of PDGF-BB spiked in 5% serum using the biotin-linked DNA-aptamer-conjugated gFFNP;

FIG. 6B is a graph showing a linear correlation based on a linearized form of the Langmuir absorption isotherm.

### DETAILED DESCRIPTION

The present invention provides a protein nanoparticle in which a fluorescent protein is fused to a self-assembled protein and located at an outside of the fusion protein.

The protein nanoparticle according to the present invention is a spherical protein particle having a nanometer-sized diameter, which includes a fusion protein of the self-assembled protein and the fluorescent protein.

In the present invention, the self-assembled protein refers to a protein, a subunit of a protein or a peptide which has a self-organized structure or pattern and forms a complex when a plurality of proteins, subunits of a protein, or peptides are assembled. Since such a self-assembled protein may form a nanoparticle of a protein without separate manipulation, it may be preferably used to manufacture the protein nanoparticle according to the present invention.

When the self-assembled protein is fused with the fluorescent protein, the fluorescent protein is adjusted to be located at an outside of the fusion protein. The protein nanoparticle according to the present invention uses a fluorescent protein to detect a target material. If the fluorescent protein is expressed to be located inside during self-assembly of the protein, fluorescence intensity is decreased. For this reason, a kind or a fused region of the self-assembled protein used as a fusion partner of the fluorescent protein, or a method of fusing or expressing a protein, may be suitably selected for the fluorescent protein to be located at the outside of the fusion protein.

In one embodiment of the present invention, the protein nanoparticle may further include a linker peptide between the self-assembled protein and the fluorescent protein. The linker peptide makes a distance between the self- assembled protein and the fluorescent protein. Generally, it is known that a fluorescence quenching phenomenon occurs when fluorescent materials are disposed within 1 to 10 nm of each other. As the linker peptide used in the present invention widens a space between fluorescent proteins, the linker peptide is considered to inhibit such fluorescence quenching, and thus increase the fluorescence intensity.

The linker peptide may have a length capable of ensuring a suitable space between the fluorescent proteins. Thus, the length of the linker peptide may vary depending on a kind and a size of the fluorescent protein. For example, the linker peptide may be a peptide composed of 5 to 20, preferably, 5 to 15 amino acids.

In one embodiment of the present invention, the linker peptide may include glycine. The linker peptide of the present invention may be, but is not limited to, a peptide having any one of amino acid sequences represented by SEQ ID NOS: 3 to 7.

Meanwhile, today, a variety of research into in vivo imaging using a magnetic nanoparticle is going on, but toxicity of the magnetic nanoparticle has been constantly an issue in its safety. However, the protein nanoparticle of the present invention is a biocompatible material which may be decomposed after being used in vivo, and thus there is no toxicity problem caused by remaining nanoparticles after the in vivo imaging. Particularly, when a protein nanoparticle in which a human- derived self-assembled protein is fused with a fluorescent protein is prepared and used for the vivo imaging, there are no safety and toxicity problems. Therefore, in one embodiment of the present invention, the self-assembled protein may be a human-derived self-assembled protein. In the present invention, the human-derived self-assembled protein or the protein nanoparticle including the same is considered to further include a humanized self-assembled protein or a humanized protein nanoparticle.

In one embodiment of the present invention, the self-assembled protein may be, but is not limited to, ferritin. The ferritin is composed of 24 identical medium and light chain protein subunits, and forms a spherical hollow shell in vivo due to a self-assembly characteristic.

In one embodiment, the self-assembled protein may be a ferritin heavy chain (FTN-H) protein.

In an example of the present invention, a ferritin medium chain protein having an amino acid sequence of SEQ ID NO: 1 was used as a self-assembled protein. The amino acid sequence of SEQ ID NO: 1 means a sequence at the 79^{th} to 85^{th} positions from the N terminal end of the sequence of NCBI Accession No: NP_{—}002023.2. In addition, the ferritin protein may be represented by an amino acid sequence of SEQ ID NO: 2.

The fluorescent protein fused to the ferritin may be, but is not limited to, fused to the C-terminal end of the ferritin. The fluorescent protein fused with the ferritin may be very useful to detect a target material because the fluorescent protein is located on a surface of the protein nanoparticle and thus provides high fluorescence intensity.

Meanwhile, in the protein nanoparticle according to the present invention, the fluorescent protein may be any known in the art. In one embodiment, the fluorescent protein may be a green fluorescent protein (GFP), modified green fluorescent protein (mGFP), enhanced green fluorescent protein (eGFP), red fluorescent protein (RFP, DSRed), enhanced red fluorescent protein (ERFP), blue fluorescent protein (BFP), enhanced blue fluorescent protein (eBFP), yellow fluorescent protein (YFP), enhanced yellow fluorescent protein (eYFP), cobalt fluorescent protein (CFP), or enhanced cobalt fluorescent protein (eCFP). In addition, Cy, Alexa fluor dye, a quantum dot, and a chemiluminescent reporter may be also added to or substituted with the fluorescent protein to be used as an optical reporter.

In one embodiment of the present invention, the protein nanoparticle may have an amino acid sequence of SEQ ID NO: 8 or 9. The protein nanoparticle of

SEQ ID NO: 8 is a protein nanoparticle in which ferritin is fused with a fluorescent protein, eGFP (GenBank: ADQ73885.1, SEQ ID NO: 10), and the protein nanoparticle of SEQ ID NO: 9 is a protein nanoparticle in which ferritin is fused with a fluorescent protein, DsRed (GenBank: BAE53441.1, SEQ ID NO: 11). These protein nanoparticles are synthesized by additionally inserting the linker according to the present invention between the ferritin and the fluorescent protein nanoparticle.

In one embodiment of the present invention, the fluorescent protein fused to the protein nanoparticle may be eGFP, which specifically may have an amino acid sequence of SEQ ID NO: 12. The amino acid sequence is a sequence in which serine located at the 175^{th} position from the N terminal end of the sequence of SEQ ID NO: 10 is substituted with cysteine. Since a DNA aptamer can covalently bind to the mutated 175^{th} amino acid, cysteine, the amino acid sequence is preferable to binding of the aptamer.

In a specific aspect of the present invention, the inventors confirmed that recombinant fluorescent protein nanoparticles (FTN-H::Linker::Fluorescent protein nanoparticle; Examples 3 and 4), which were constructed by inserting a linker peptide between a FTN-H nanoparticle and a fluorescent protein, displayed considerably enhanced fluorescent emission and particle stability, compared with those constructed by fusing a self-assembled FTN-H nanoparticle with a fluorescent protein (Examples 1 and 2). These results indicate that the degree of fluorescent emission was considerably high, for example, approximately 20 or more times that of a single fluorescent protein (Control 1) not fused with a FTN-H nanoparticle. As a result, it is estimated that, since the protein nanoparticle according to the present invention has superior fluorescence intensity and stability, the protein nanoparticle may be useful as an optical reporter for in vitro or in vivo imaging.

In addition, the present invention provides another protein nanoparticle in which a probe is bound to the above-described protein nanoparticle. The probe serves to bind to a target material and detect the target material in response to a fluorescence signal of the protein nanoparticle according to the present invention, and a kind of the probe is not specifically limited. It is clear to those of skill in the art that the kind of the probe will also vary depending on a kind of the target material.

In one embodiment, the probe may be an aptamer. A kind of the aptamer to be fused may vary depending on the kind of a target material, which is well known in the art.

The aptamer binding to the protein nanoparticle according to the present invention serves to target a target material and control a distance between the fluorescent proteins, thereby further increasing fluorescence intensity of the fluorescent protein on the surface of the protein nanoparticle.

In a specific aspect of the present invention, the inventors synthesized a protein nanoparticle (DNA aptamer-gFFNP in Examples 6 and 8) having an DNA aptamer (SEQ ID NO: 14) bound to a surface of a FTN-H::Linker::Fluorescent protein nanoparticle, the aptamer being specific to a platelet-derived growth factor B-chain homodimer (PDGF-BB) known as a cancer marker. Compared with a FTN-H::Linker::Fluorescent protein particle, the aptamer-conjugated protein nanoparticle had higher fluorescence intensity. It is believed that the negatively charged PDGF-BB-specific aptamer conjugated to a surface of FTN-H controlled a distance between the fluorescent proteins, and thus the fluorescence intensity of the fluorescent protein on the surface of FTN-H was further increased.

In one embodiment of the present invention, the aptamer-conjugated protein nanoparticle may have an amino acid sequence of SEQ ID NO: 13, but the present invention is not limited thereto.

Moreover, the present invention provides a biosensor including the above- described protein nanoparticle and a method of detecting a target material using the protein nanoparticle.

The biosensor including the protein nanoparticle may be used for qualitative or quantitative analysis of a target material, or diagnosis of various diseases, and may include additional components conventionally used in the biosensor.

The present invention also provides a method of detecting a target material including confirming whether a probe of the protein nanoparticle described above reacts with the target material. The protein nanoparticle may allow a reaction of the probe with the target material to be detected using a fluorescence signal. The detection method may be used in vitro or in vivo without limitation. Particularly, the protein nanoparticle according to the present invention may ensure structural stability, biocompatibility and safety, and thus may be very useful to detect a target material in vivo, that is, in vivo imaging.

In a specific aspect of the present invention, the inventors covalently attached a DNA aptamer to a surface of gFFNP, and the aptamer-conjugated gFFNP was used as a reporter for three-dimensional signal amplification for sandwich analysis (dual-site binding assay) based on an aptamer of PDGF-BB which is a biomarker for diagnosing cancer. As a result, it was confirmed that the aptamer- conjugated gFFNP had superior sensitivity in a PBS aqueous solution or serum including PDGF-BB.

In addition, since the protein nanoparticle in which a fluorescent protein is fused to a self-assembled protein has superior fluorescence intensity and structural stability, compared with a monomer-type fluorescent protein, it may be applied in a conventional fluorescent protein-based detection method, for example, enzyme-linked immunosorbent assay (ELISA).

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

The terminology used herein to describe embodiments of the invention is not intended to limit the scope of the invention. The articles “a,” “an,” and “the” are singular in that they have a single referent, however the use of the singular form in the present document should not preclude the presence of more than one referent. In other words, elements of the invention referred to in the singular may number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof. Herein, the term “and/or” includes any and all combinations of one or more referents.

Exemplary embodiments of the present invention will be described in detail below with reference to the appended drawings. Elements of the exemplary embodiments are consistently denoted by the same reference numerals throughout the drawings and detailed description, and description of the same elements will not be reiterated.

### EXAMPLES

### Examples 1 to 4

### Manufacture of Recombinant Fluorescent Protein Nanoparticle

To synthesize fluorescent protein-fused protein nanoparticles according to the present invention (recombinant eGFP, DsRed and gFFNP), PCR amplification was performed using libraries and primers listed in Table 1, and thereby 6 clones were generated (refer to Tables 1 and 3). Here, the PCR reactions were carried out under the conditions: 1) pre-denaturation for 5 minutes at 94° C.; 2) denaturation for 30 seconds at 94° C., 3) annealing for 30 seconds at 52° C. and 4) extension for 30 seconds at 72° C., (here, 2) to 4) were repeated 30 cycles), and then 5) reacting for 5 minutes at 72° C. A total reaction volume was set to 20 μl.

Here, linkers inserted between fluorescent proteins and ferritin nanoparticles were listed in Table 2, and in the following Examples, the linker represented by SEQ ID NO: 3 was used (refer to Table 2).

TABLE 1

| | Clone | PCR Conditions |
|---|---|---|
| 1 | N-NdeI-(hFTN-H)-XhoI-C | Forward Primer (SEQ ID NO: 15) |
| | | Reverse Primer (SEQ ID NO: 16) |
| | | Template: human liver cDNA library |
| | | (clontech, USA) |
| 2 | N-NdeI-hexahistidine- | Forward Primer (SEQ ID NO: 17) |
| | (eGFP)-HindIII-C | Reverse Primer (SEQ ID NO: 18) |
| | | Template: pEGFP plasmid |
| | | (clontech, USA) |
| 3 | N-XhoI-eGFP-hexahistidine- | Forward Primer (SEQ ID NO: 19) |
| | HindIII-C | Reverse Primer (SEQ ID NO: 20) |
| | | Template: pEGFP plasmid |
| | | (clontech, USA) |
| 4 | N-XhoI-G3SG3TG3SG3- | 1^{st} PCR |
| | eGFP-H6-HindIII-C | SEQ ID NO: 22, SEQ ID NO: 23 |
| | | Template: pEGFP plasmid |
| | | (clontech, USA) |
| | | 2^{nd} PCR |
| | | SEQ ID NO: 21, SEQ ID NO: 23 |
| | | Template: 1^{st} PCR product |
| 5 | N-XhoI-(DsRed)- | Forward Primer (SEQ ID NO: 24) |
| | hexahistidine-HindIII-C | Reverse Primer (SEQ ID NO: 25) |
| | | Template: pDsRed-Monomer Vector |
| | | (clontech, USA) |
| 6 | N-XhoI-G3SG3TG3SG3- | 1^{st} PCR |
| | DsRed-H6-HindIII-C | SEQ ID NO: 27, SEQ ID NO: 28 |
| | | Template: pDsRed-Monomer Vector |
| | | (clontech, USA) |
| | | 2^{nd} PCR |
| | | SEQ ID NO: 26, SEQ ID NO: 28 |
| | | Template: 1^{st} PCR product |

TABLE 2

| | Sequence |
|---|---|
| SEQ ID NO: 3 | GGGSGGGSGGGSGGG |
| SEQ ID NO: 4 | GGGGG |
| SEQ ID NO: 5 | GGGSGGGTGGGSGGG |
| SEQ ID NO: 6 | GGGGSGGGGT |
| SEQ ID NO: 7 | GGGGSGGGGS |

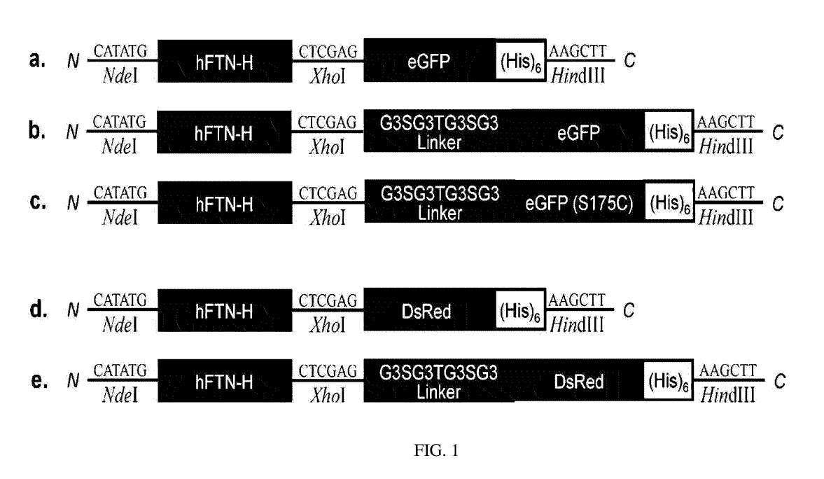

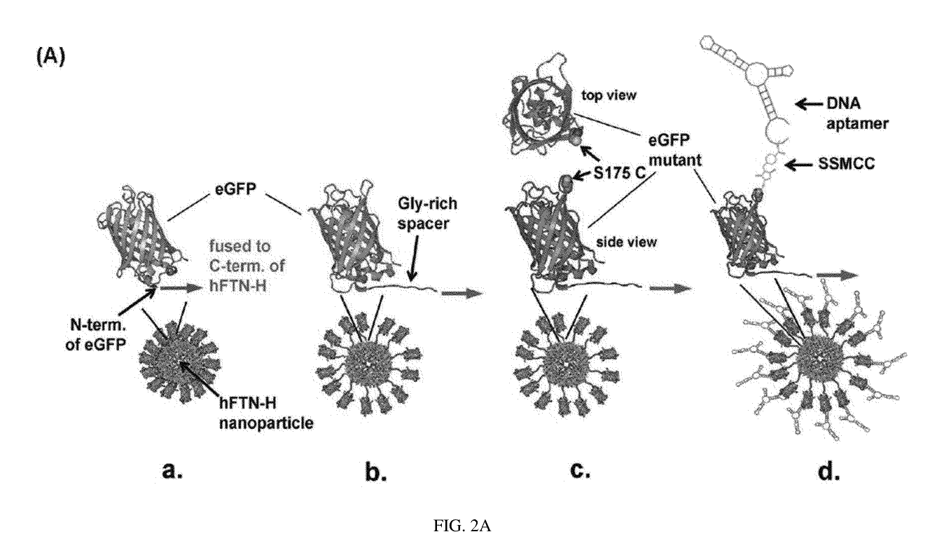

The gene clones were ligated into pT7-7 plasmid (Novagen, USA), and thereby various expression vectors were constructed as shown in FIG. 1. The pT7-7 vector was ligated with respective clones listed in Table 2, thereby constructing pT7-GFP, pT7-FTN-GFP, pT7-FTN-RED, pT7-FTH-LNK-GFP and pT7-FTH-LNK-RED expression vectors (refer to Table 3). Cloning of the hFTN-H gene was the same as described in the conventional art, Korean Patent No. 10-0772491.

TABLE 3

| | Expression Vector | |
|---|---|---|
| Control 1 | pT7-GFP | pT7 Plasmid Vector + Clone 2 |
| Example 1 | pT7-FTN-GFP | pT7 Plasmid Vector + Clone 1 + |
| | | Clone 3 |
| Example 2 | pT7-FTN-RED | pT7 Plasmid Vector + Clone 1 + |
| | | Clone 5 |
| Example 3 | pT7-FTH-LNK-GFP | pT7 Plasmid Vector + Clone 1 + |
| | | Linker + Clone 4 |
| Example 4 | pT7-FTH-LNK-RED | pT7 Plasmid Vector + Clone 1 + |
| | | Linker + Clone 6 |

After sequencing of the constructed vectors was completed, each expression vector was transformed into E. coli BL21(DE3) [F_ompThsdSB(rB_mB_)], and then transformants having ampicillin resistance were finally selected.

Methods of gene expression induced by the isopropyl β-D-1-thiogalactopyranoside (IPTG), purification of a recombinant fluorescent ferritin nanoparticle and transmission electron microscopy (TEM) imaging of the purified protein nanoparticle were the same as disclosed in the previous reports conducted by the inventors (Park, J. S, et al., J. Nat. Nanotechnol. 2009; Lee, S. H. et al., FASEB J. 2007; Lee, J. H. et al., J. Adv. Funct. Mater. 2010; Seo, H. S. et al., Adv. Funct. Mater. 2010; Ahn, J. Y. et al., J. Nucleic Acids Res. 2005).

### Examples 5 to 9

### Conjugation of DNA Aptamers to eGFP, gFFNP and Cy3

Induction of eGFP Site-Directed Mutagenesis for Conjugating DNA Aptamer

In the present invention, a method of chemically conjugated a DNA aptamer to a surface of a protein nanoparticle using sulfosuccinimidyl 4-(N-maleimidomethyl)cyclohexane-1-carboxylate (SSMCC) was used. The SSMCC forms an amine-thiol heterofunctional cross-linker to covalently coupling the protein nanoparticle with the DNA aptamer. However, since two cysteines having a thiol functional group of eGFP were located in the eGFP structure, it was difficult to conjugate the aptamer to the position where the cysteines were located. For this reason, the inventors substituted serine at the 175^{th} position at an external loop of eGFP with cysteine using site-specific mutation, and selected the position of the cysteine as a DNA aptamer conjugation position.

To conjugate the DNA aptamer to eGFP and gFFNP (FFNP fused to eGFP, Example 1), the 175^{th} residue of eGFP, serine, was mutated into cysteine (Ser175Cys), and primers used herein were as follows (refer to Table 4).

TABLE 4

| | Sequence (5′-3′) |
|---|---|
| SEQ ID NO: 29 | AACATCGAGGACGGCTGCGTGCAGCTCGCC |
| (Forward Primer) | |
| SEQ ID NO: 30 | GGCGAGCTGCACGCAGCCGTCCTCGATGTT |
| (Reverse Primer) | |

* manufactured by Genotech, Daejon, (South Korea), Tm = 86.1° C.

The site-directed mutagenesis was performed using the optical procedure described in previous report by the inventors (Ahn, J. Y.et al., J. Nucleic Acids Res. 2005). After DNA gel purification and sequencing, the E. coli BL21 (DE3) was transformed with expression vectors capable of respectively encoding the site- specifically mutated eGFP (Ser175Cys) and gFFNP+eGFP (Ser175Cys), and then transformants having ampicillin resistance were finally selected. Methods of expressing and purifying the recombinant gene and analyzing a TEM image of a fluorescent ferritin nanoparticle were the same as described above.

Synthesis of DNA Aptamer Specific to PDGF-BB

To confirm if the aptamer conjugated to the fluorescent protein nanoparticle (gFFNP) according to the present invention can be applied to a diagnostic system, an aptamer specific to PDGF-BB which is generally known as a marker for detecting various cancers such as lung, breast, and stomach cancers was fused to gFFNP (Ariad, S. et al., Breast Cancer Res. Treat, 1991; Lubinus, M. et al., M. J. Biol. Chem, 1994).

An aptamer represented by SEQ ID NO: 14, which is specific to the PDGF- BB and has high compatibility, was synthesized, and then three kinds of aptamers were obtained by fusing an amine, Cy3 and biotin to the aptamer, respectively (refer to Table 5).

TABLE 5

| | | |
|---|---|---|
| 1 | Amine-modified | 5′NH₂-(CH₂)₆- |
| | DNA Aptamer | CACAGGCTACGGCACGTAGAGCATCACCATGATCCTGTGT-3′ |
| 2 | Cy3-modified | 5′Cy3- |
| | DNA Aptamer | CACAGGCTACGGCACGTAGAGCATCACCATGATCCTGTGT-3′ |
| 3 | Biotin-modified | 5′biotin-(CH₂)₆- |
| | DNA Aptamer | CACAGGCTACGGCACGTAGAGCATCACCATGATCCTGTGT-3′ |

* manufactured by Genotech, Daejon (South Korea)

Synthesis of DNA Aptamer-Conjugated gFFNP (DNA Aptamer-gFFNP)

First, to activate the previously constructed aptamer, 40 μl of the aptamer diluted in distilled water to have a concentration of 100 μM was reacted with 60 μl of a dimethylformamide (DMF) solution including 100 μl of PBS buffer [137 mM NaCl, 2.7 mM KCl, 10 mM Na₂HPO₄, 2 Mm KH₂PO₄, pH 7.4] and 2 mg of SSMCC (Pierce, Rockford, Ill.) at 35° C. for 1 hour. Here, unreacted excess SSMCC was removed using a QIAEX II Gel Extraction kit (QIAGEN, Duesseldorf, Germany).

Afterward, 100 μl of 1M dithiothreitol (DTT) was added to 1 ml of gFFNP consisting of hFTN-H, a linker peptide and the mutated eGFP, and the resulting mixture was incubated for 30 minutes at 35° C. to remove any internanoparticle disulfide bridges. Then, the solution volume was reduced to 500 μl using ultrafiltration (Amicon Ultra 100K, Millipore, Billerica, Mass.). The SSMCC-activated DNA aptamer and the volume-reduced gFFNPs were combined and incubated in the dark for 2 hours at a room temperature to conjugate the PDGF-BB-specific aptamer to the gFFNP.

The unconjugated/free DNA aptamers to gFFNP were separated from the DNA aptamer-gFFNP conjugates (DNA aptamer-gFFNP) using ultrafiltration (Amicon Ultra 100K). The retentate buffer was exchanged to an anion-exchange buffer [20 mM [bis(2-hydroxyethyl)amino]tris(hydroxymethyl)methane (Bis-Tris), pH 6.0] using ultrafiltration described above. Afterward, to remove the free gFFNP and purify the DNA aptamer-gFFNP, anion exchange chromatography using a Q sepharose fast flow bead column (GE Healthcare, Buckinghamshire, U.K.) was performed.

After the chromatography, an NaCl concentration gradually increased from 0 to 0.7 M (pH 6.0) for elution, and the buffer for the purified DNA aptamer-gFFNP conjugates was exchanged to storage buffer [150 mM NaCl, 36.4 mM KH2PO4, 63.6 mM K2HPO4, 5 mM EDTA, pH 7.5].

Synthesis of DNA Aptamer-Conjugated-eGFP (DNA Aptamer-eGFP and Cy3)

In the case of DNA-aptamer-conjugated eGFP, a DNA aptamer was conjugated to eGFP by the same method as used for the DNA aptamer-gFFNP in Example <2-1>. However, there is a difference from Example <2-1>in that nickel affinity chromatography (QUAGEN) was used to remove a unconjugated/free DNA aptamer. Except for that, the purification steps were the same as for the DNA aptamer-gFFNP described above.

A DNA concentration of the DNA aptamer-eGFP conjugates or DNA aptamer-gFFNP conjugates was estimated by measuring an absorbance at 260 nm. A concentration of the protein nanoparticle was measured using a Bradford method, using the predetermined correlation: absorbance from the sample containing one ferritin particle and 24 eGFP monomers (Biovision, Mountain View, Calif.) was regarded as an absorbance from one nanoparticle of gFFNP.

TABLE 6

| | Expression Vector |
|---|---|
| Example 5 | pT7-FTH-LNK-GFP (S175C) |
| Example 6 | pT7-FTH-LNK-GFP (S175C)-Aptamer 1 |
| Example 7 | pT7-FTH-LNK-Aptamer 2 |
| Example 8 | pT7-FTH-LNK-GFP (S175C)-Aptamer 3 |
| Example 9 | pT7-GFP (S175C)-Aptamer 1 |

### Experimental Example 1

### Measurement of Emission Intensity of Fluorescent Protein Nanoparticle

<1-1>Emission Intensity of eGFP Fluorescent Protein Nanoparticle (gFFNP)

The inventors analyzed the emission intensities of the fluorescent protein nanoparticles constructed in the Examples and the TEM images of the particles. Specifically, to measure the emission intensity of each particle, Tecan (GeNios) was used, and the method was the same as described in Kim K R et al., Biochem Biophys Res Commun. 2011 408(2):225-9.

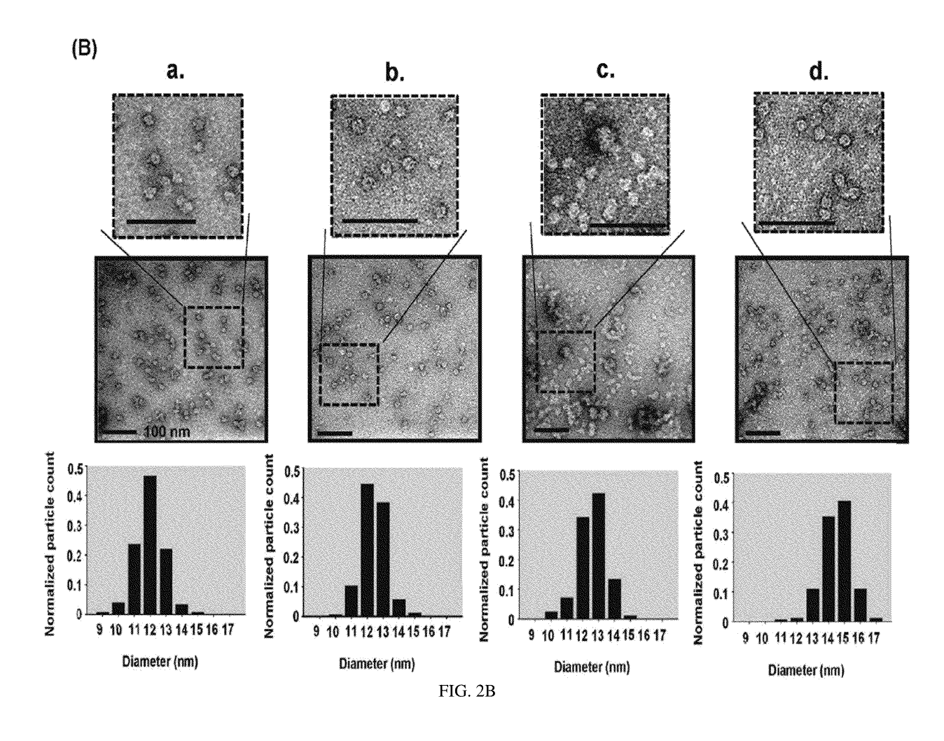

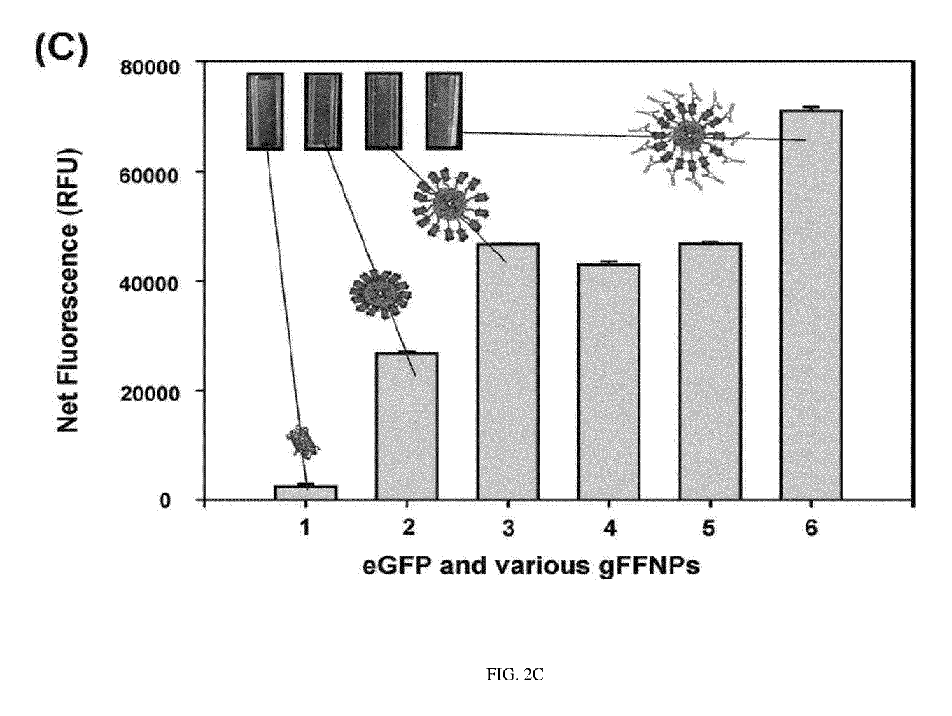

Specifically, among the fluorescent nanoparticles of Examples 1 to 9, the particles shown in FIG. 2A (Examples 1, 2, 5 and 6), which were eGFP-fused proteins emitting green fluorescence, were used. In FIG. 2A, (a) is a schematic diagram of a protein in which eGFP was fused with hFTN-H, (b) is a schematic diagram of a fusion protein in which a linker peptide was linked between eGFP and hFTN-H, (c) is a schematic diagram of eGFP(Ser175Cys)-hFTN-H in which the 175^{th} serine of eGFP was mutated into cysteine, and (d) is a schematic diagram of a fusion protein in which an aptamer was linked to the eGFP(Ser175Cys)-hFTN-H (refer to FIG. 2A).

According to the results of evaluating a degree of emission of the fluorescent ferritin nanoparticle, it can be seen that a degree of fluorescent emission of the protein in which hFTN-H was fused to eGFP (Example 1) was approximately 11 times greater than an eGFP single fluorescent protein (Control 1) (Bar 2 of FIG. 2C).

However, since 24 eGFPs were linked to one ferritin, this result is believed to indicate that fluorescence emission increased by approximately 50% (FIG. 2C, Bar 1—eGFP single fluorescent protein, and Bar 2—the protein in which hFTN-H is fused to eGFP in Example 1). (In analysis of FIG. 2C, to compare the fluorescence intensity of single eGFP with the fluorescence intensity emitted from single gFFNP, the number of protein nanoparticles in a gFFNP solution was adjusted the same as the number of DsRed protein molecules in a DsRed solution.) It is estimated that such a result is caused by fluorescence quenching occurring because eGFP is closed to gFFNP. It is known that the fluorescent quenching generally occurs when the fluorescent materials are present within 1 to 10 nm of each other.

In addition, Bar 3 in FIG. 2C shows the result of fluorescence emission of the fusion protein of Example 2, in which a linker peptide (G3SG3TG3SG3; length: approximately 4.5 nm; SEQ ID NO: 3) was inserted into the C terminal end of hFTN-H and the N terminal end of eGFP (refer to FIGS. 1 and 2A). When the linker peptide was inserted between eGFP and hFTN-H, the fluorescence emission was approximately 1.74 times higher than that of the fluorescent protein not including a linker peptide in Example 1, which indicates that it is approximately 20 times higher than that of eGFP. It is believed that such a result was obtained by reducing a quenching effect due to increases in flexibility and solubility of the protein induced by the linker peptide.

In addition, Bar 6 of FIG. 2C shows a degree of fluorescence emission of the fluorescent protein nanoparticle to which the DNA aptamer was fused in Example 6. The fluorescence emission was 29 times higher than that of only eGFP (Bar 1 of FIG. 2C), which indicates that it is also increased by approximately 50% based on the degree of emission of the protein having a linker peptide in Example 2. It is believed that such a result occurred by reducing the quenching effect because a spatial distance between eGFPs located on a surface of gFFNP was properly maintained due to the electrostatic repulsion of a negative charged nucleic acid of the conjugated DNA aptamer.

Moreover, FIG. 2C shows a result of comparing degrees of fluorescence emission between the protein nanoparticle (FTH-LNK-GFP (S175C)) in Example 5 and the particle constructed by treating the fluorescent protein nanoparticle in Example 5 with DTT. From FIG. 2C, it was observed that mutation of the fluorescent protein itself and the DTT treatment used in the fusion of the aptamer did not have an influence on the degree of fluorescence emission. It was also confirmed from the TEM images and histograms that the ferritin nanoparticles having uniform sizes were spherical.

<1-2>DsRed Fluorescent Protein Nanoparticle (rFFNP)

An experiment was performed by the same method as described in Experimental Example <1-1>, except that fluorescence intensities of fluorescent ferritin nanoparticles (rFFNPs and hFTN-H-DsRed) to which the fluorescent material DsRed, not eGFP, bonded, were measured. Here, the intensities were compared whether a linker peptide was or was not present between the ferritin and the DsRed fluorescent material. That is, characteristics of the nanoparticles in Examples 3 and 4 were compared. The methods of measuring fluorescence emission and analyzing TEM images were the same as described in Experimental Example <1-1>.

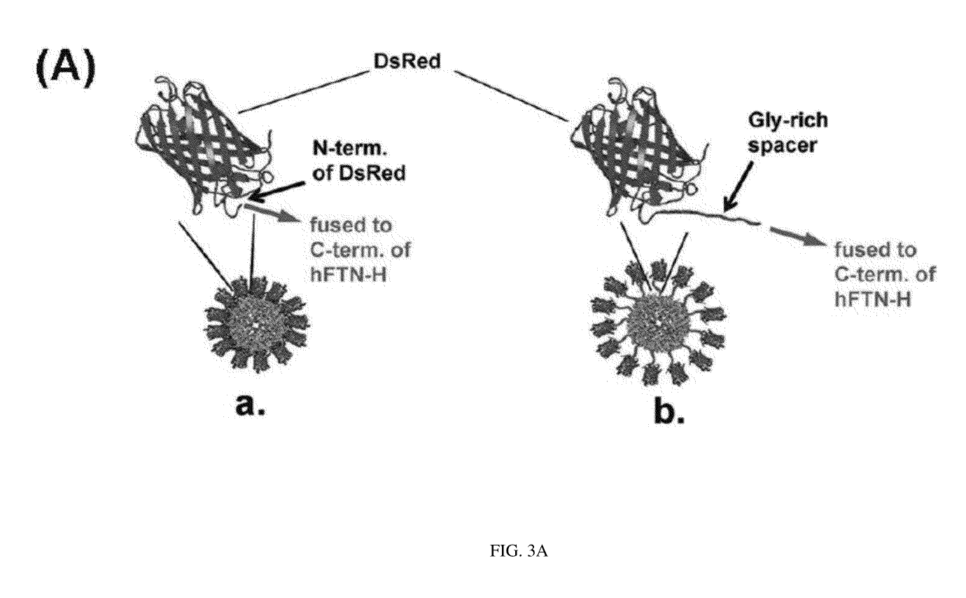

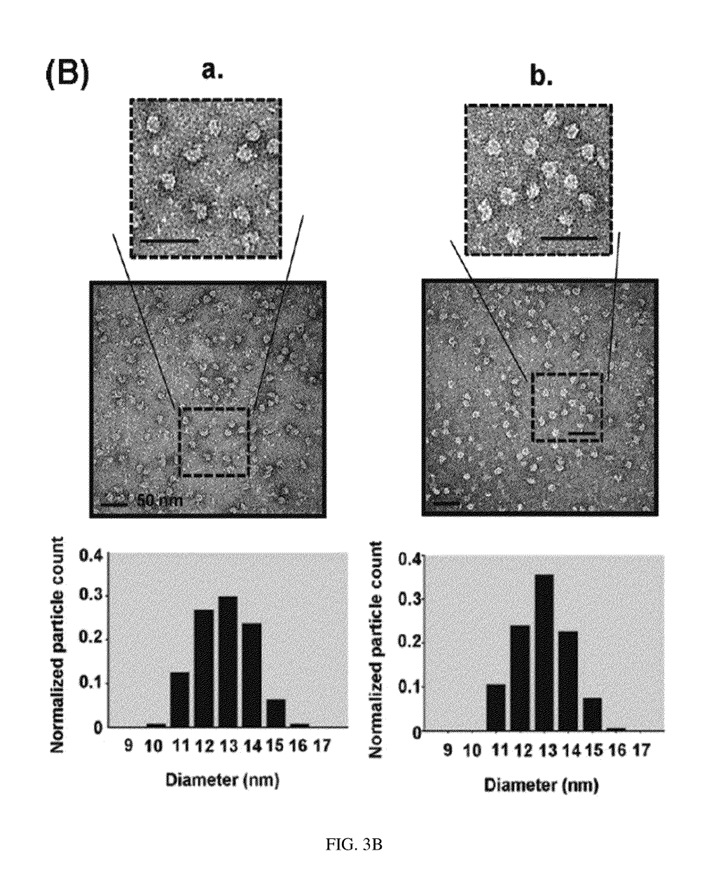

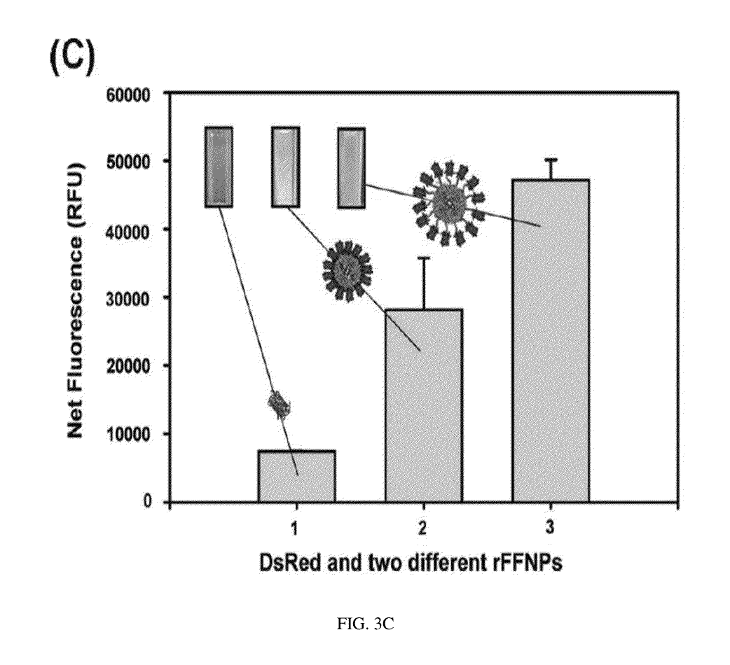

Bar 2 of FIG. 3C shows the result of observing the protein nanoparticle in Example 3, in which a linker peptide was not present between the ferritin nanoparticle and the DsRed fluorescent material. Compared with DsRed (Bar 1 of FIG. 3C) not fused with ferritin, fluorescence emission increased approximately 4 times. That is, it was observed that such a result was obtained by a considerably stronger quenching effect between DsRed fluorescent proteins of linker peptide-free rFFNP than that of eGFP of gFFNP (in analysis of FIG. 3C, to compare fluorescence intensities emitted from single rFFNP and single DsRed, a number of the protein nanoparticles present in the rFFNP solution was adjusted to be the same as the DsRed protein molecules in the DsRed solution).

Bar 3 of FIG. 3C shows the result of a degree of fluorescence emission of the protein nanoparticle in which the same linker peptide as that inserted into gFFNP of Experimental Example <1-1>was inserted between hFTN-H and DsRed in Example 4, and the fluorescence emission increased by 68% based on rFFNP into which a linker peptide was not inserted. In addition, from TEM images and histograms shown in FIG. 3B, it was confirmed that the ferritin nanoparticles having uniform sizes were spherical.

Consequently, from the results shown in FIGS. 2 and 3, it was confirmed that the linker peptide inserted between hFTN-H and the fluorescent protein (eGFP or DsRed) was a key factor in reducing the quenching effect decreasing the fluorescent degree from FFNP.

### Experimental Example 2

### Measurement of Stability of Fluorescent Protein Nanoparticle

Stability of the fluorescent protein nanoparticle according to the present invention was confirmed through results of spot measurement performed at intervals of 2 days for 16 days. The eGFP and gFFNP samples were not constantly exposed to an excitation wavelength throughout an analysis period. That is, the samples were exposed to the excitation wavelength at intervals of 2 days.

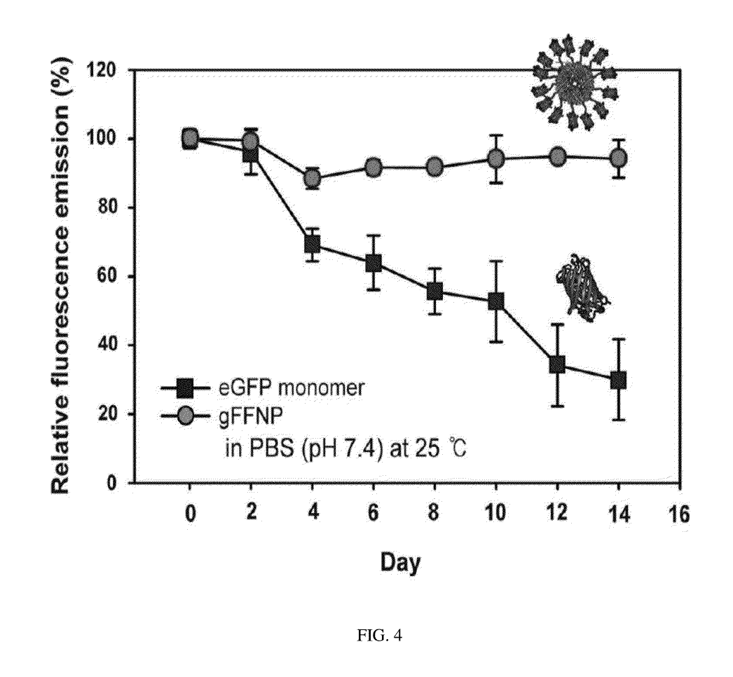

According to the analysis of long-term stability at a mild temperature condition (e.g., 25° C.), despite a stable β-barrel structure of eGFP, emission intensity of eGFP decreased by 60% of an initial level within 2 weeks, but 90% or more of the initial level of fluorescence emission of gFFNP was retained during the same period as eGFP. Such a result indicates that the stability of gFFNP according to the present invention was significantly enhanced (refer to FIG. 4).

### Experimental Example 3

### Aptamer-Based Biomolecular Detection Method Using Fluorescent Protein Nanoparticle

Through the Experimental Examples, it was confirmed that the fluorescent protein nanoparticle developed according to the present invention was superior in fluorescence intensity, stability and sensitivity when fused with a DNA aptamer. Accordingly, the inventors performed an experiment for detecting PDGF-BB in a PBS aqueous solution or diluted serum using a fluorescent ferritin nanoparticle fused with a PDGF-BB-specific aptamer to confirm if the DNA aptamer-fluorescent ferritin nanoparticle is useful as a probe.

Specifically, aptamer-based sandwich analysis was performed using gFFNPs respectively fused with amine, Cy3 and biotin, which were synthesized in Examples 6 to 8, as a probe. Here, the experiment was performed using gFFNP because eGFP had higher brightness and photostability than DsRed (Shaner, N. et al., Nat. Methods 2005).

<3-1>96-Well PDGF-BB Detection Method Using DNA Aptamer-gFFNP Probe

Before immobilization of the biotin-modified DNA aptamer probes(capture probes) the Costar high-binding 96-well plate(Corning Inc., Corning, N.Y.) was incubated with 100 ng of streptavidin (New England Biolabs, Hitchin, Herts, England) in PBS buffer [137 mM NaCl, 2.7 mM KCl, 10 mM Na₂HPO₄, 2 mM KH₂PO₄, pH 7.4] including at 4° C. for 12 hours. Afterward, 20 μl of a biotin-modified DNA aptamer (10 nM) was incubated in the 96-well plate for 1 and a half hours. The plate was then washed with the PBS buffer for 5 minutes.

150 μl volume of analyte sample containing PDGF-BB (1 fM to 10 nM) (Sigma-Aldrich, St. Louis, Mo.) in either PBS buffer or healthy human serum (5%) was added to each well and incubated at 37° C. for 1 h.

Afterward, the plate was washed with the PBS buffer for 5 minutes, then 35 ul of DNA aptamer-gFFNP (5 μg/mL) in the storage buffer was added to each well and incubated at 37° C. for 1 hour. After the three consecutive washing steps followed by the addition of 50 μl of PBS buffer to each well, the fluorescence signals were measured using a microplate reader (485 nm excitation/535 nm emission; Tecan, GENios).

<3-2>96-Well PDGF BB Detection Method Using eGFP- and Cy3-Fused Proteins

An experiment for detecting PDGF BB from a 96-well plate using DNA aptamer-conjugated eGFP and DNA aptamer-conjugated Cy3 was performed by the same method as described above, except that fluorescent proteins fused with eGFP and Cy3, instead of a ferritin nanoparticle (gFFNP), were respectively fused with the DNA aptamer.

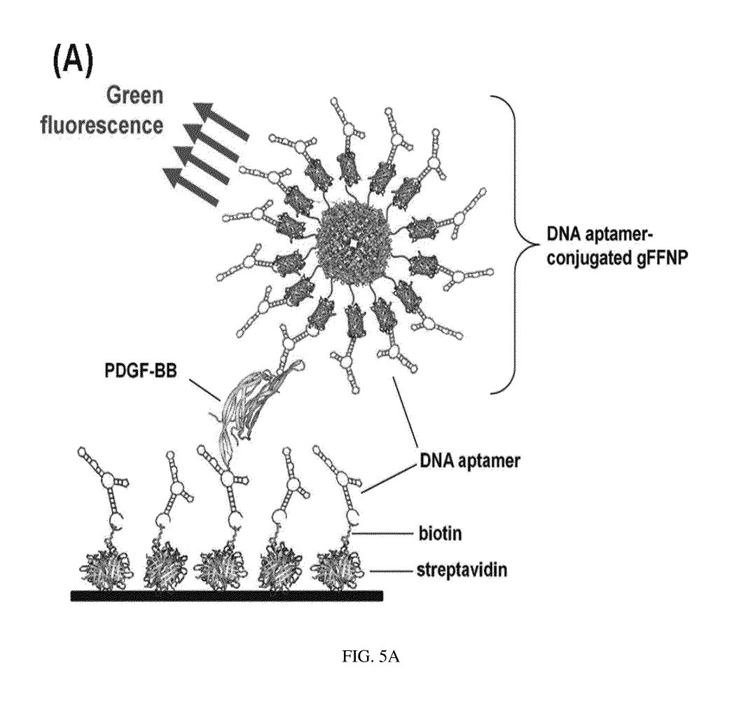

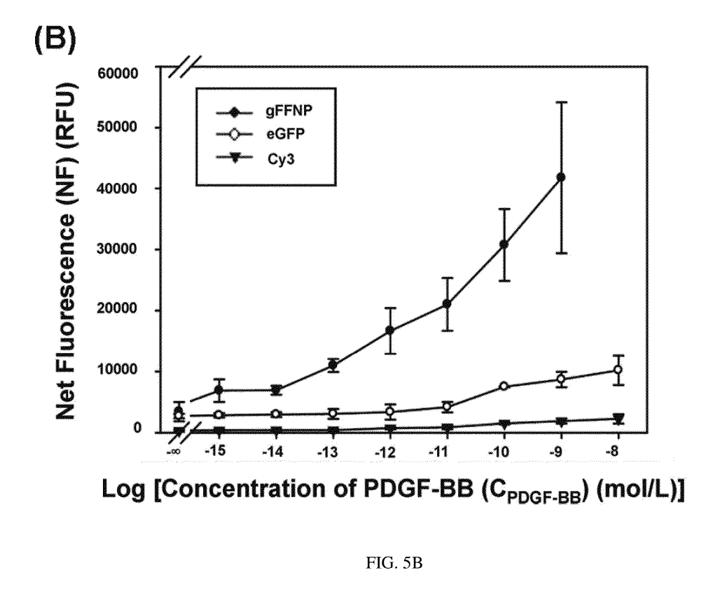

As a result of the analysis shown in FIG. 5B, compared with the biotin DNA aptamer-gFFNP, fluorescence signals of the DNA aptamer-eGFP and Cy3 were considerably low in an entire concentration range of PDGF BB. As a result, the graph of FIG. 5B is a typical Langmuir-isotherm curve, which means that the signal is rarely linear over the concentration range, and converges to a saturation value at a high solute concentration. It is estimated that the result of signal saturation is due to saturation of capture probes, inhibition of solute binding to capture probes bysolute already bound, binding-site competition, etc.

According to the typical Langmur-isotherm model (a linearized form of the absorption isotherm curve, C/NF=C/NF_{satd}+KD/NF_{satd}, wherein C, NF, NF_{satd} and KD represent PDGF-BB concentration, net fluorescence (sensor signal), saturated net fluorescence and dissociation constant, respectively), as clearly shown in FIG. 5, signals are all linear at sufficiently dilute solute concentration.

On the Basis of the linearized form of the Langmur absorption isotherm and the linear curve of FIG. 5C, the dissociation constants (KD) were determined by a PDGF-BB analysis method using each of the DNA-aptamer-conjugated gFFNP, eGFP, and Cy3, and the results are shown in Table 7.

TABLE 7

| | DNA-aptamer- | DNA-aptamer- | DNA-aptamer- |
|---|---|---|---|
| | conjugated FFNPs | conjugated eGFP, | conjugated Cy3 |
| Dissociation | 6.0 × 10¹⁴ | 4.0 × 10¹¹ | 5.0 × 10¹¹ |
| Constant | | | |
| (KD) | | | |

*unit: mol · L⁻¹

It was determined from these results that, compared with the DNA-aptamer-conjugated eGFP and Cy3 reporters, the three-dimensional structure of DNA-aptamer-gFFNP, i.e., 24 DNA aptamers that are attached per single spherical gFFNP, may give more efficient access of gFFNPs to the target marker protein, PDGF-BB, and allow more sensitive detection.

That is, it is estimated that the detection method using the DNA aptamer-gFFNP according to the present invention may decrease a detection limit from a picomolar level to a nanomolar level in the eGFP-based analysis, and thus may be used in an aptamer-based analysis method.

In addition, compared with what is estimated from the results shown in FIG. 2C, i.e., that DNA aptamer-conjugated gFFNP is approximately 29 times more sensitive than eGFP in an aqueous solution, the difference in fluorescent signals between gFFNP- and eGFP-based analyses was observed to be small. It is believed that such a result is obtained because a self-quenching phenomenon more severely happens on the surface than in the aqueous solution. Moreover, since eGFP bound to gFFNP has a higher local density than eGFP not bound to gFFNP when exposed to a surface, it is believed that gFFNP had a more extreme self-quenching phenomenon than eGFP when exposed to the two-dimensional surface.

<3-3>DNA-Aptamer-gFFNP Analysis Method Using Biological Sample

To confirm whether the analysis method using DNA aptamer-gFFNP according to the present invention has superior sensitivity and a detection effect with respect to a biological sample, unlike Experimental Example <3-1>, serum including DPGF-BB as an analysis subject was used.

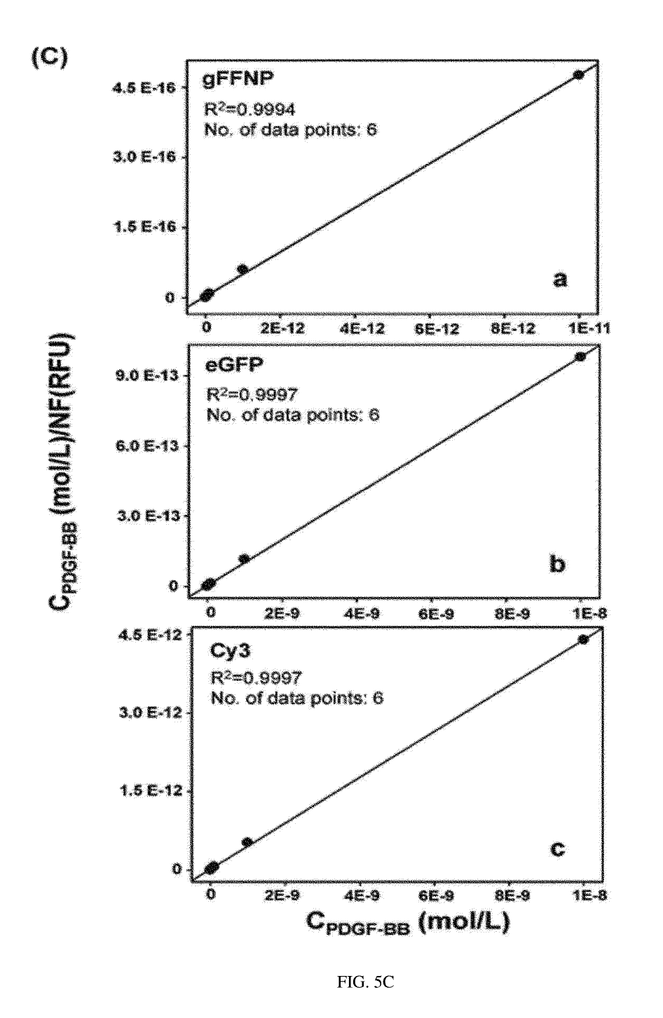

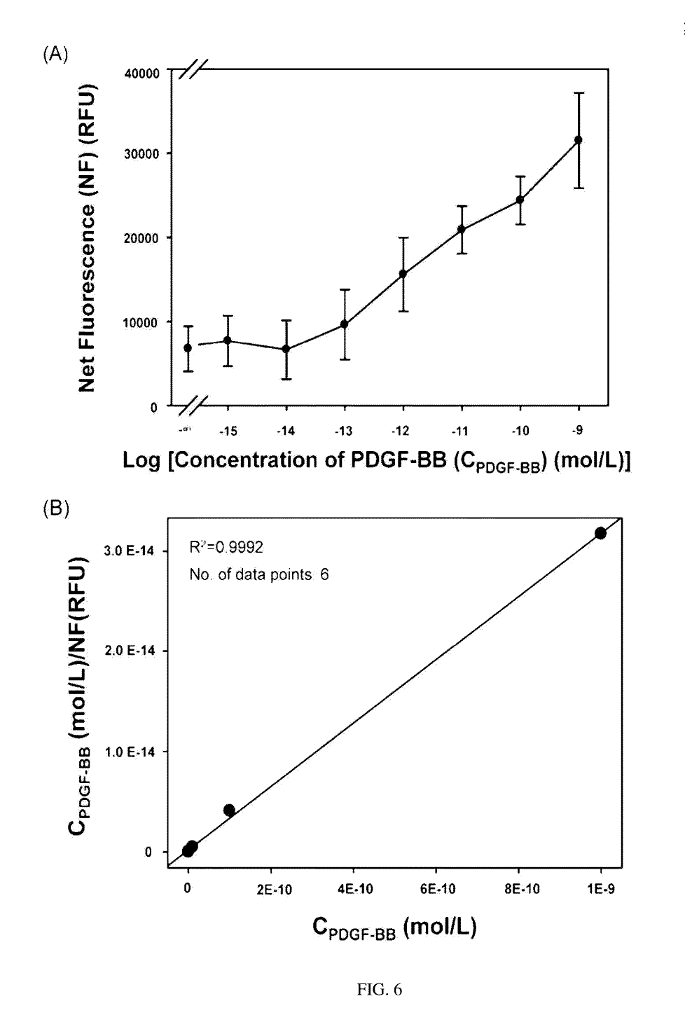

Consequently, as shown in FIG. 6, through the same DNA aptamer-based analysis as in Experimental Example <3-1>, a PDGF-BB spiked in the diluted serum (5%) of a healthy human was also successfully detected with a bit higher LOD (1 to 10 Pm of PDGF-BB), demonstrating that the assay could be properly performed even in the biological environment (refer to FIG. 6). FIG. 6A is a typical Langmur-isotherm curve, and FIG. 6B shows that the signals are all linear at a diluted concentration of the PDGF-BB.

What is claimed is:

1. A protein nanoparticle in which a fluorescent protein is fused to a self- assembled protein, the fluorescent protein being located at an outside of a fusion protein.

2. The protein nanoparticle of claim 1, further comprising a linker peptide between the self-assembled protein and the fluorescent protein.

3. The protein nanoparticle of claim 1, wherein the self-assembled protein is a human-derived self-assembled protein.

4. The protein nanoparticle of claim 1, wherein the self-assembled protein is ferritin.

5. The protein nanoparticle of claim 1, wherein the self-assembled protein is a ferritin medium chain protein.

6. The protein nanoparticle of claim 2, wherein the linker peptide comprises glycine.

7. The protein nanoparticle of claim 2, wherein the linker peptide is a peptide having one of amino acid sequences represented by SEQ ID NOS: 3 to 7.

8. The protein nanoparticle of claim 1, wherein the fluorescent protein is selected from the group consisting of a green fluorescent protein (GFP), modified green fluorescent protein (mGFP), enhanced green fluorescent protein (eGFP), red fluorescent protein (RFP, DSRed), enhanced red fluorescent protein (ERFP), blue fluorescent protein (BFP), enhanced blue fluorescent protein (eBFP), yellow fluorescent protein (YFP), enhanced yellow fluorescent protein (eYFP), cobalt fluorescent protein (CFP), and enhanced cobalt fluorescent protein (eCFP).

9. The protein nanoparticle of claim 1, wherein the protein nanoparticle has an amino acid sequence represented by SEQ ID NO: 8 or 9.

10. The protein nanoparticle of claim 1, wherein the fluorescent protein is eGFP having an amino acid sequence represented by SEQ ID NO: 12.

11. The protein nanoparticle of claim 1, wherein a probe is conjugated to the protein nanoparticle.

12. The protein nanoparticle of claim 11, wherein the probe is an aptamer.

13. A biosensor comprising the protein nanoparticle of claim 11.

14. A method of detecting a target material, comprising:
confirming whether the probe of the protein nanoparticle of claim 11 reacts with a target material.

15. The method of claim 14, wherein the method is performed in vitro or in vivo.


SEQUENCE LISTING (30 sequences)

SEQ ID NO: 1 - protein, 7 aa, Homo sapiens
GRIFLQD

SEQ ID NO: 2 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 3 - protein, 15 aa, Artificial Sequence
  Linker peptide 1
GGGSGGGSGGGSGGG

SEQ ID NO: 4 - protein, 5 aa, Artificial Sequence
  Linker peptide 2
GGGGG

SEQ ID NO: 5 - protein, 15 aa, Artificial Sequence
  Linker peptide 3
GGGSGGGTGGGSGGG

SEQ ID NO: 6 - protein, 10 aa, Artificial Sequence
  Linker peptide 4
GGGGSGGGGT

SEQ ID NO: 7 - protein, 10 aa, Artificial Sequence
  Linker peptide 5
GGGGSGGGGS

SEQ ID NO: 8 - protein, 444 aa, Artificial Sequence
  Recombinant fluorescent nanoparticle (eGFP)
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESLEGGGSGGGTGGGSGGGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHH

SEQ ID NO: 9 - protein, 430 aa, Artificial Sequence
  Recombinant fluorescent nanoparticle (DsRed)
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNESLEGGGSGGGTGGGSGGGDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQHHHHHH

SEQ ID NO: 10 - protein, 238 aa, Artificial Sequence
  eGFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - protein, 224 aa, Artificial Sequence
  DsRed
DNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQ

SEQ ID NO: 12 - protein, 238 aa, Artificial Sequence
  eGFP mutant (S175C)
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGCVQLADHYQQNTPIGDGPVLLPDNHYLSTQSALSKDPNEKRDHMVLLEFVTAAGITLGMDELYK

SEQ ID NO: 13 - protein, 444 aa, Artificial Sequence [sequence omitted]

SEQ ID NO: 14 - DNA, 40 bp, Artificial Sequence
  PDGF-BB aptamer
cacaggctacggcacgtagagcatcaccatgatcctgtgt

SEQ ID NO: 15 - DNA, 21 bp, Artificial Sequence
  Forward primer for Clone1
catatgacgaccgcgtccacc

SEQ ID NO: 16 - DNA, 24 bp, Artificial Sequence
  Reverse primer for Clone1
ctcgaggctttcattatcactgtc

SEQ ID NO: 17 - DNA, 47 bp, Artificial Sequence
  Forward primer for Clone2
catatgcatcatcaccaccatcatctcgaggtgagcaagggcgagga

SEQ ID NO: 18 - DNA, 24 bp, Artificial Sequence
  Reverse primer for Clone2
aagcttttacttgtacagctcgtc

SEQ ID NO: 19 - DNA, 23 bp, Artificial Sequence
  Forward primer for Clone3
ctcgaggtgagcaagggcgagga

SEQ ID NO: 20 - DNA, 42 bp, Artificial Sequence
  Reverse primer for Clone3
aagcttttagtgatggtgatggtgatgcttgtacagctcgtc

SEQ ID NO: 21 - DNA, 45 bp, Artificial Sequence
  Forward primer1 for Clone4
ctcgagggtggcggaagtgggggaggcactggaggtggcagcggc

SEQ ID NO: 22 - DNA, 42 bp, Artificial Sequence
  Forward primer2 for Clone4
actggaggtggcagcggcggtggggtgagcaagggcgaggag

SEQ ID NO: 23 - DNA, 42 bp, Artificial Sequence; identical to SEQ ID NO: 20
  Reverse primer for Clone4

SEQ ID NO: 24 - DNA, 25 bp, Artificial Sequence
  Forward primer for Clone5
ctcgaggacaacaccgaggacgtca

SEQ ID NO: 25 - DNA, 43 bp, Artificial Sequence
  Reverse primer for Clone5
aagcttttagtgatggtgatggtgatgctgggagccggagtgg

SEQ ID NO: 26 - DNA, 45 bp, Artificial Sequence; identical to SEQ ID NO: 21
  Forward primer1 for Clone6

SEQ ID NO: 27 - DNA, 43 bp, Artificial Sequence
  Forward primer2 for Clone6
actggaggtggcagcggcggtggggacaacaccgaggacgtca

SEQ ID NO: 28 - DNA, 43 bp, Artificial Sequence; identical to SEQ ID NO: 25
  Reverse primer for Clone6

SEQ ID NO: 29 - DNA, 30 bp, Artificial Sequence
  Forward primer for mutagenesis
aacatcgaggacggctgcgtgcagctcgcc

SEQ ID NO: 30 - DNA, 30 bp, Artificial Sequence
  Reverse primer for mutagenesis
ggcgagctgcacgcagccgtcctcgatgtt